(19) United States
(12) Patent Application Publication
(10) Pub. No.: US 2010/0239685 A1
(43) Pub. Date: Sep. 23, 2010

(54) COMPOUND FOR TREATMENT OR PREVENTION OF PROSTATE-RELATED DISEASES AND PHARMACEUTICAL COMPOSITION OF COLON DELIVERY SYSTEM CONTAINING THE SAME

(76) Inventors: Taehwan Kwak, Gyeonggi-do (KR); Sang-Ku Yoo, Gyeonggi-do (KR); Myung-gyu Park, Gyeonggi-do (KR)

Correspondence Address: BIRCH STEWART KOLASCH & BIRCH, PO BOX 747, FALLS CHURCH, VA, 22040-0747, US

(21) Appl. No.: 12/515,014

(22) PCT Filed: Nov. 26, 2007
(86) PCT No.: PCT/KR07/06012

§ 371 Date: May 15, 2009

(30) Foreign Application Priority Data
Nov. 27, 2006 (KR) 10-2006-0117685
Nov. 1, 2007 (KR) 10-2007-0111183

(51) Int. Cl. A61K 31/39; A61K 31/353; A61K 31/343; A61K 31/352; A61K 31/382; A61K 31/381; A61K 9/52; A61P 13/08; A61P 5/24

(52) U.S. Cl. 424/501; 514/454; 514/468; 514/453; 514/437; 514/443; 514/434

(57) ABSTRACT

Provided is a naphthoquinone-based compound represented by Formula 1 or 2 having therapeutic effect on the treatment and/or prevention of prostate and/or testicle (seminal glands)-related diseases, and to a pharmaceutical composition of intestinal delivery system containing the same.

### FIELD OF THE INVENTION

The present invention relates to a naphthoquinone-based compound having therapeutic effect on the treatment and/or prevention of prostate and/or testicle (seminal glands)-related diseases, and to a pharmaceutical composition of intestinal delivery system containing the same.

### BACKGROUND OF THE INVENTION

Prostate is a walnut-shape organ that surrounds the distal urethra in men and is located just below the urinary bladder where it produces mucous substances. The only function of the prostate is to protect semen and produce seminal fluid for breeding. The prostate has characteristics in that it grows larger with aging and that most mammals have the urethra for excreting urine from the bladder which passes through the prostate. Due to such anatomical characteristics of the prostate, men may sometimes have urinary excretion-related problems and induce various diseases including prostate-related diseases.

The prostate-related disease is a collective term for prostatitis and benign prostatic hyperplasia.

The prostatitis is defined as an infection or inflammation of the prostate. High fever, acheness and stiffness caused by the prostatitis are generally chronic symptoms, although there are acute cases. Thus, despite the standard therapy, the prostatitis is considered one of incurable diseases with a relatively high relapse rate. Statistically, 30% or more of men in 20s to 50s suffer from a prostate-related disease, and it is a frequently occurring disease which occupies 25% or more of the outpatients in urology. However, the full recovery rate of the disease is very low, thereby having problems that 80 to 90% of the above patients suffers recurrence.

The prostatitis is induced by inflammation of the prostate tissue, and its symptoms include conspicuously frequent micturition, thinning of urine flow, burning pain during urination, indisposed pain in the abdominal and perineal region, and serious testicle or back pain. In addition, these symptoms become intense after drinking or overworking, thereby progressing to other general symptoms of sexual dysfunction, prospermia, and fatigue.

Particularly, chronic prostatitis is a disease which frequently appears in adult men. Prostatitis has substantial relation to all prostate cancer. Even with no other peculiar symptoms, inflammation may be found through histologic examination. When suffering from the chronic prostatitis, pain commonly begins at the pelvic area and progresses to symptoms such as urinary hesitancy, impotence and sterility. A typical symptom, ‘urinary hesitancy’, associated with prostatitis leads to symptoms such as lack of sleep caused by nocturia, weak urinary stream and urinary retention.

The benign prostatic hyperplasia (BPH) refers to the increase in size of the prostate. The disease occupies the most frequent occurrence among male urinary dysfunction, and its frequency increases as men get older. Men aged 60 or older suffer from this disease relatively more often. As a result, the disease increases in its rate of occurrence in the aging society.

The benign prostatic hyperplasia, as the prostate enlarges, show symptoms including frequency of urination, especially nocturine (waking up to urinate about 2 to 4 times at night time), difficulty starting the urine flow and decreased urination force. After feeling occasional unpleasantness or oppression in the perineal region and stiffness in abdominal region, the irritative symptoms and urination symptoms that have been observed from beginning become intensed as the conditions develops to finally being unable to urinate. Thereafter, the urine amount is reduced even more, and residual urine amount are increased resulting in obstructive effects on the kidney.

The chronic prostatitis and benign prostatic hyperplasia may not show serious symptoms in many men. However, they are chronic diseases that give serious influence to the quality of life. Moreover, their diagnosis is difficult and complete cure of the diseases is also difficult. In addition, the occurrence of the prostate cancer tends to rise due to the increase in the average lifespan and westernized diet.

Thus, there is an increasingly urgent need to develop an efficient substance suitable for use as a safe drug for the substantial treatment of the prostate and/or testicle (seminal glands)-related diseases.

### SUMMARY OF THE INVENTION

Therefore, the present invention has been made to solve the above problems and other technical problems that have yet to be resolved.

As a result of a variety of extensive and intensive studies and experiments based on the facts as described above, the inventors of the present invention have confirmed that a specific naphthoquinone-based compound is effective for prevention and/or treatment of prostate and/or testicle (seminal glands)-related diseases. The present invention has been completed based on these findings. Such pharmaceutical effects are very new and have been unknown to the present.

In addition, another object of the present invention is to provide a pharmaceutical composition of an intestinal delivery system including, as an active ingredient, the naphthoquinone-based compound.

In accordance with an aspect of the present invention, the above and other objects can be accomplished by the provision of one or more selected from the compounds represented by the following Formula 1 and Formula 2, or a pharmaceutically acceptable salt, prodrug, solvate or isomer thereof, having a therapeutic effect on the treatment and prevention of prostate and/or testicle (seminal glands)-related diseases:

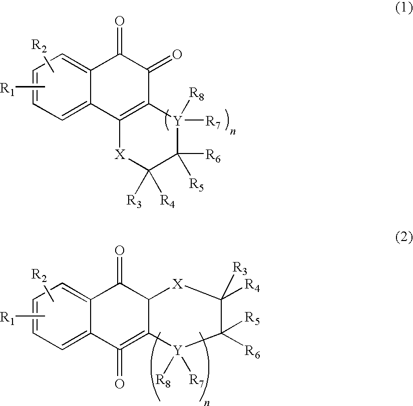

wherein

R₁ and R₂ are each independently hydrogen, halogen, hydroxyl, or C₁-C₆ lower alkyl or alkoxy, or R₁ and R₂ may be taken together to form a substituted or unsubstituted cyclic structure which may be saturated or partially or completely unsaturated;

R₃, R₄, R₅, R₆, R₇ and R₈ are each independently hydrogen, hydroxyl, amino, C₁-C₂₀ alkyl, alkene or alkoxy, C₄-C₂₀ cycloalkyl, heterocycloalkyl, aryl or heteroaryl, or two substituents of R₃ to R₈ may be taken together to form a cyclic structure which may be saturated or partially or completely unsaturated;

X is selected from a group consisting of C(R)(R′), N(R″), O and S, preferably O or S, and more preferably O, wherein R, R′ and R″ are each independently hydrogen or C₁-C₆ lower alkyl;

Y is C, S or N, with proviso that when Y is S, R₇ and R₈ are not any substituent, and when Y is N, R₇ is hydrogen or C₁-C₆ lower alkyl and R₈ are not any substituent; and

n is 0 or 1, with proviso that when n is 0, carbon atoms adjacent to n form a cyclic structure via a direct bond, or a pharmaceutically acceptable salt, prodrug, solvate or isomer thereof.

As used the present disclosure, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, tichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as arginine, lysine and guanidine, salts with organic bases such as dicyclohexylamine, N-methyl-D-glucamine, tris(hydroxymethyl)methylamine, diethanolamine, choline and triethylamine. The compound 1 or 2 in accordance with the present invention may be converted into salts thereof, by conventional methods well-known in the art.

As used herein, the term “prodrug” means an agent that is converted into the parent drug in vivo. Prodrugs are often useful because, in some situations, they may be easier to administer than the parent drug. They may, for instance, be bioavailable by oral administration, whereas the parent may be not. The prodrugs may also have improved solubility in pharmaceutical compositions over the parent drug. An example of a prodrug, without limitation, would be a compound of the present invention which is administered as an ester (the “prodrug”) to facilitate transport across a cell membrane where water-solubility is detrimental to mobility, but which then is metabolically hydrolyzed to the carboxylic acid, the active entity, once inside the cell where water solubility is beneficial. A further example of the prodrug might be a short peptide (polyamino acid) bonded to an acidic group, where the peptide is metabolized to reveal the active moiety.

As an example of such prodrug, the pharmaceutical compounds in accordance with the present invention can include a prodrug represented by Formula 1a below:

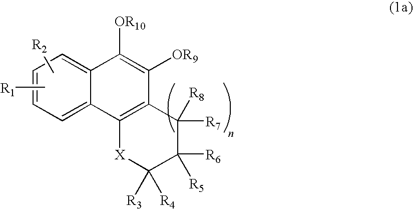

wherein,

R₁, R₂, R₃, R₄, R₅, R₆, R₇, R₈, X and n are as defined in Formula 1.

R₉ and R₁₀ are each independently —SO₃—Na⁺ or substituent represented by Formula A below or a salt thereof,

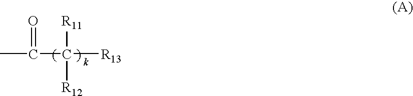

wherein,

R₁₁ and R₁₂ are each independently hydrogen or substituted or unsubstituted C₁-C₂₀ linear alkyl or C₁-C₂₀ branched alkyl

R₁₃ is selected from the group consisting of substituents i) to viii) below:

i) hydrogen;

ii) substituted or unsubstituted C₁-C₂₀ linear alkyl or C₁-C₂₀ branched alkyl;

iii) substituted or unsubstituted amine;

iv) substituted or unsubstituted C₃-C₁₀ cycloalkyl or C₃-C₁₀ heterocycloalkyl;

v) substituted or unsubstituted C₄-C₁₀ aryl or C₄-C₁₀ heteroaryl;

vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;

vii) substituted or unsubstituted carboxyl;

viii) —OSO₃—Na⁺;

k is selected from the 0˜20, with proviso that when k is 0, R₁₁ and R₁₂ are not anything, and R₁₃ is directly bond to a carbonyl group.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof, that has the same chemical formula or molecular formula but is optically or sterically different therefrom. D type optical isomer and L type optical isomer can be present in the Formula 1 or Formula 2, depending on the R₃˜R₈ types of substituents selected.

Unless otherwise specified, the term “naphthoquinone-based compound” or “compound of Formula 1 or 2” is intended to encompass a compound per se, and a pharmaceutically acceptable salt, prodrug, solvate and isomer thereof.

As used herein, the term “alkyl” refers to an aliphatic hydrocarbon group. The alkyl moiety may be a “saturated alkyl” group, which means that it does not contain any alkene or alkyne moieties. Alternatively, the alkyl moiety may also be an “unsaturated alkyl” moiety, which means that it contains at least one alkene or alkyne moiety. The term “alkene” moiety refers to a group in which at least two carbon atoms form at least one carbon-carbon double bond, and an “alkyne” moiety refers to a group in which at least two carbon atoms form at least one carbon-carbon triple bond. The alkyl moiety, regardless of whether it is substituted or unsubstituted, may be branched, linear or cyclic.

As used herein, the term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

As used herein, the term “aryl” refers to an aromatic substituent group which has at least one ring having a conjugated pi (π) electron system and includes both carbocyclic aryl (for example, phenyl) and heterocyclic aryl (for example, pyridine) groups. This term includes monocyclic or fused-ring polycyclic (i.e., rings which share adjacent pairs of carbon atoms) groups.

As used herein, the term “heteroaryl” refers to an aromatic group that contains at least one heterocyclic ring.

Examples of aryl or heteroaryl include, but are not limited to, phenyl, furan, pyran, pyridyl, pyrimidyl and triazyl.

R₁, R₂, R₃, R₄, R₅, R₆, R₇ and R₈ in Formula 1 in accordance with the present invention may be optionally substituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino including mono and di substituted amino, and protected derivatives thereof. Further, substituents of R₁₁, R₁₂ and R₁₃ in the Formula 1a may be also substituted as defined in above, and when substituted, they can be substituted as the substituents mentioned above.

Among compounds of Formula 1, preferred are compounds of Formulas 3 and 4 below.

Compounds of Formula 3 are compounds wherein n is 0 and adjacent carbon atoms form a cyclic structure (furan ring) via a direct bond therebetween and are often referred to as “furan compounds” or “furano-o-naphthoquinone derivatives” hereinafter.

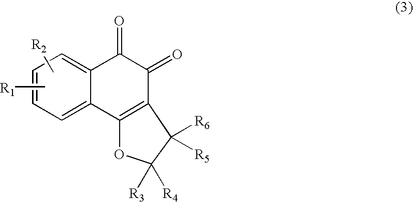

Compounds of Formula 4 are compounds wherein n is 1 and are often referred to as “pyran compounds” or “pyrano-o-naphthoquinone” hereinafter.

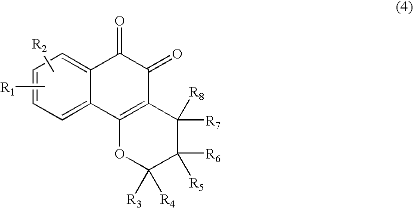

Among the furan compounds of Formula 3, preferred are compounds of Formula 3a wherein R₁, R₂ and R₄ are hydrogen, or compounds of Formula 3b wherein R₁, R₂ and R₆ are hydrogen.

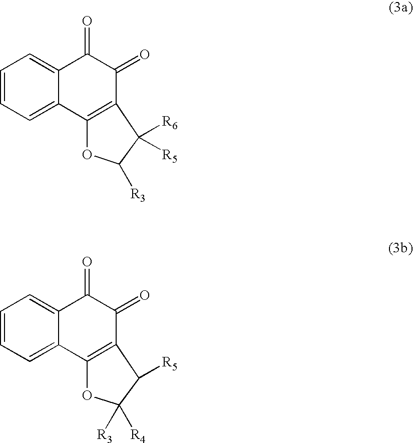

Further, among the furan compounds of Formula 3, particularly preferred are compounds below.

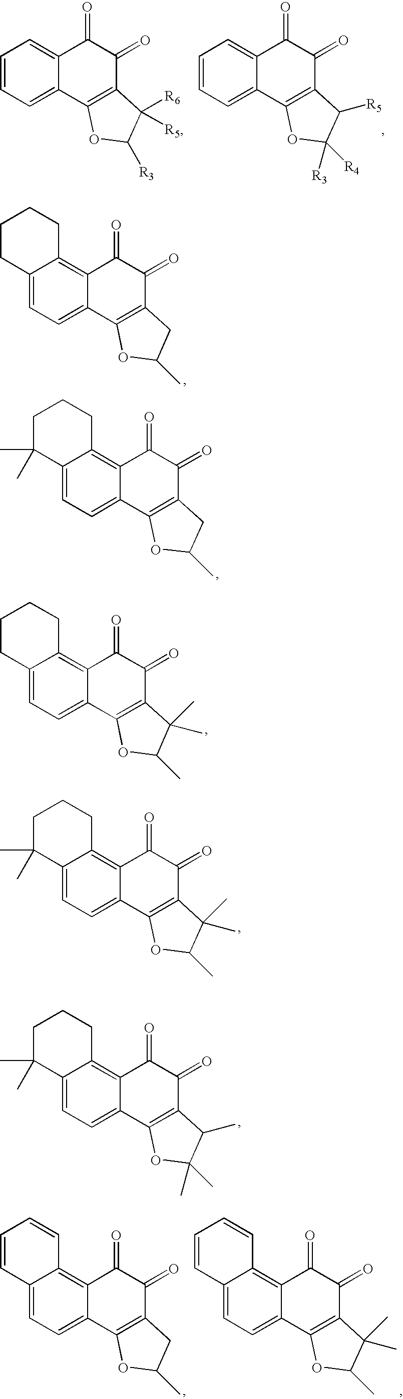

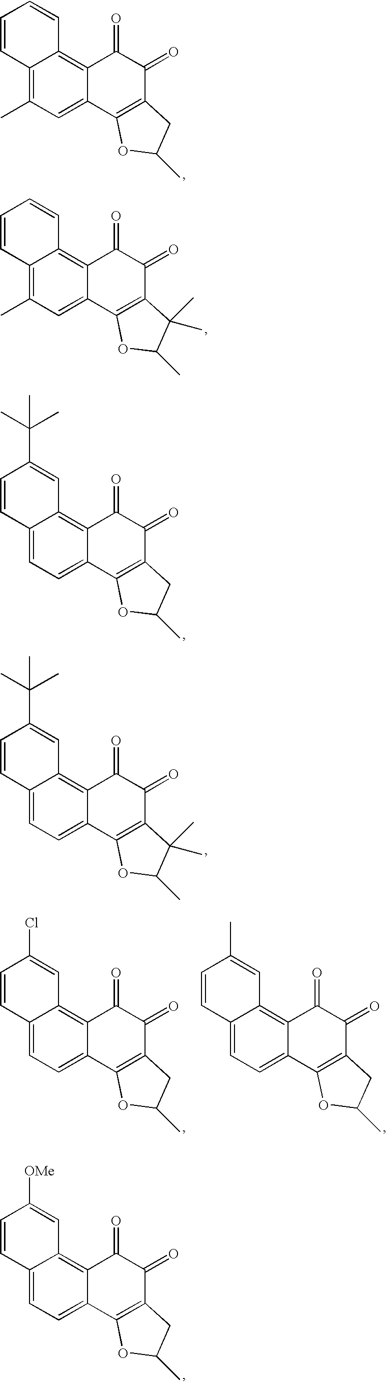

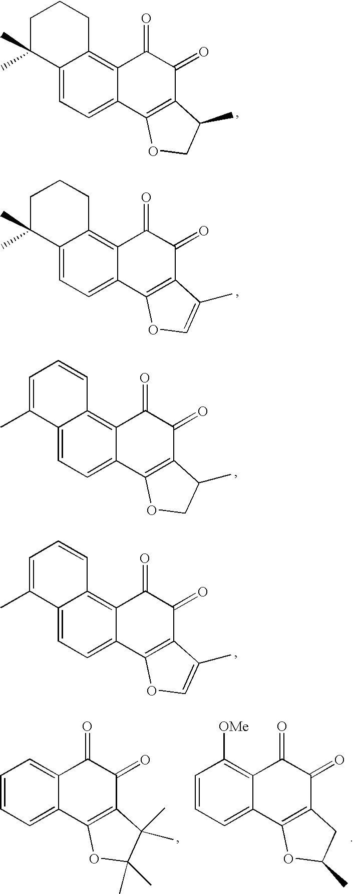

Further, among the pyran compounds of Formula 4, particularly preferred are compounds of Formula 4a wherein R₁, R₂, R₅, R₆, R₇ and R₈ are respectively hydrogen, or compounds of Formula 4b or Formula 4c wherein R₁ and R₂ are taken together to form a cyclic structure which is substituted or unsubstituted.

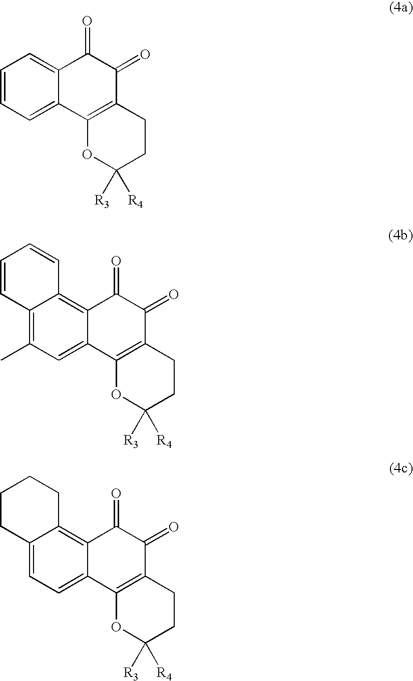

Further, among the compounds of Formula 2, preferred are compounds of Formulas 2a and 2b, but are not limited thereto.

Compounds of Formula 2a below are compounds wherein n is 0 and adjacent carbon atoms form a cyclic structure via a direct bond therebetween and Y is C.

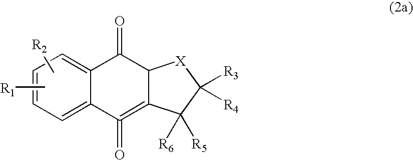

Compounds of Formula 2b below are compounds wherein n is 1 and Y is C in Formula 2.

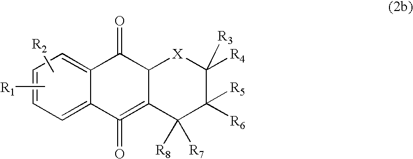

In the Formula 2a or 2b, R₁, R₂, R₃, R₄, R₅, R₆, R₇, R₈ and X are as defined in Formula 2.

Effective substance which exerts therapeutic effect on the treatment and/or prevention of prostate and/or testicle (seminal glands)-related diseases in the present invention is often referred to as “active ingredient” hereinafter.

### Preparation of Active Ingredient

In the pharmaceutical composition in accordance with the present invention, compounds of Formula 1 or Formula 2, as will be illustrated hereinafter, can be prepared by conventional methods known in the art and/or various processes which are based upon the general technologies and practices in the organic chemistry synthesis field. The preparation processes described below are only exemplary ones and other processes can also be employed. As such, the scope of the instant invention is not limited to the following processes.

In general, tricyclic naphthoquinone (pyrano-o-naphthoquinone and furano-o-naphthoquinone) derivatives can be synthesized by two methods mainly. One is to derive cyclization reaction using 3-allyl-2-hydroxy-1,4-naphthoquinone in acid catalyst condition, as the following β-lapachone synthesis scheme.

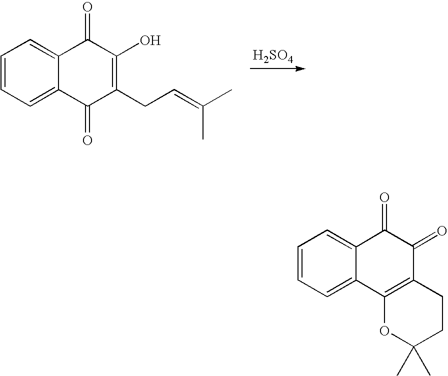

That is, 3-allyloxy-1,4-phenanthrenequinone can be obtained by deriving Diels-Alder reaction between 2-allyloxy-1,4-benzoquinone and styrene or 1-vinylcyclohexane derivatives and dehydrating the resulting intermediates using oxygen present in the air or oxidants such as NaIO₄ and DDQ. By further re-heating the above compound, 2-allyl-3-hydroxy-1,4-phenanthrenequinone of Lapachole form can be synthesized via Claisen rearrangement.

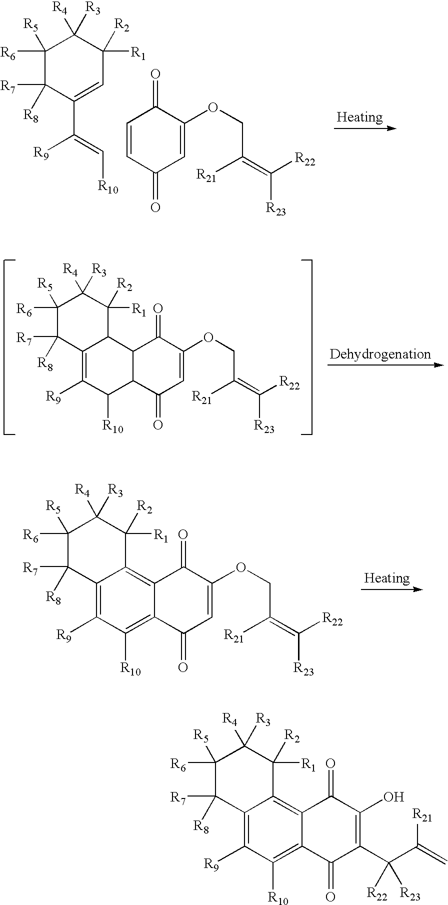

When the thus obtained 2-allyl-3-hydroxy-1,4-phenanthrenequinone is ultimately subjected to cyclization in an acid catalyst condition, various 3,4-phenanthrenequinone-based or 5,6,7,8-tetrahydro-3,4-phenanthrenequinone-based compounds can be synthesized. In this case, 5 or 6-cyclic cyclization occurs depending on the types of substituents (R₂₁, R₂₂, R₂₃ in the above formula) represented in the above formula, and also they are converted to the corresponding, adequate substituents (R₁₁, R₁₂, R₁₃, R₁₄, R₁₅, R₁₆ in the below formula).

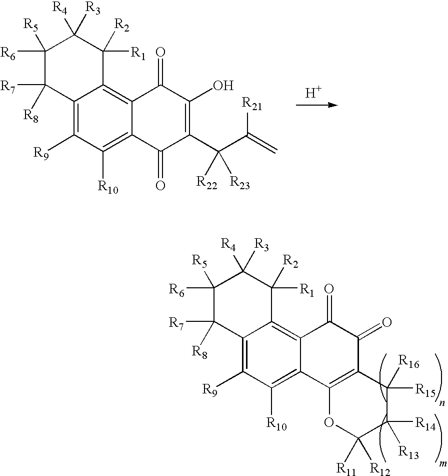

Further, 3-allyloxy-1,4-phenanthrenequinone is hydrolyzed to 3-oxy-1,4-phenanthrenequinone, in the condition of acid (H⁺) or alkali (OH⁻) catalyst, which is then reacted with various allyl halides to synthesize 2-allyl-3-hydroxy-1,4-phenanthrenequinone by C-alkylation. The thus obtained 2-allyl-3-hydroxy-1,4-phenanthrenequinone derivatives are subject to cyclization in the condition of acid catalyst to synthesize various 3,4-phenanthrenequinone-based or 5,6,7,8-tetrahydro-3,4-naphthoquinone-based compounds. In this case, 5 or 6-cyclic cyclization occurs depending on the types of substituents (R₂₁, R₂₂, R₂₃ in the above formula) represented in the above formula, and also they are converted to the corresponding, adequate substituents (R₁₁, R₁₂, R₁₃, R₁₄, R₁₅, R₁₆ in the below formula).

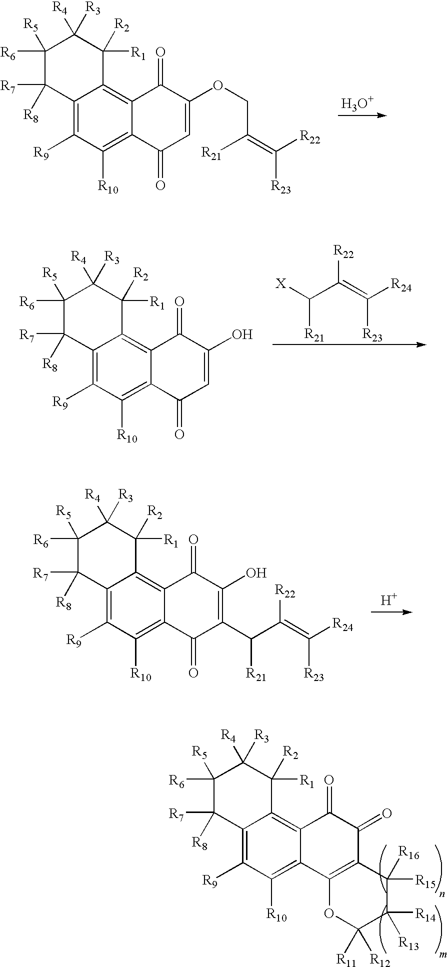

However, compounds in which substituents R₁₁ and R₁₂ are simultaneously hydrogen cannot be obtained by acid-catalyzed cyclization. These derivatives are obtained on the basis of a method reported by J. K. Snyder et al (Tetrahedron Letters 28 (1987), 3427-3430), more specifically, by first obtaining furanobenzoquinone introduced furan ring by cyclization, and then obtaining tricyclic phenanthroquinone by cyclization with 1-vinylcyclohexene derivatives, followed by reduction via hydrogen-addition. The above synthesis process can be summarized as follows.

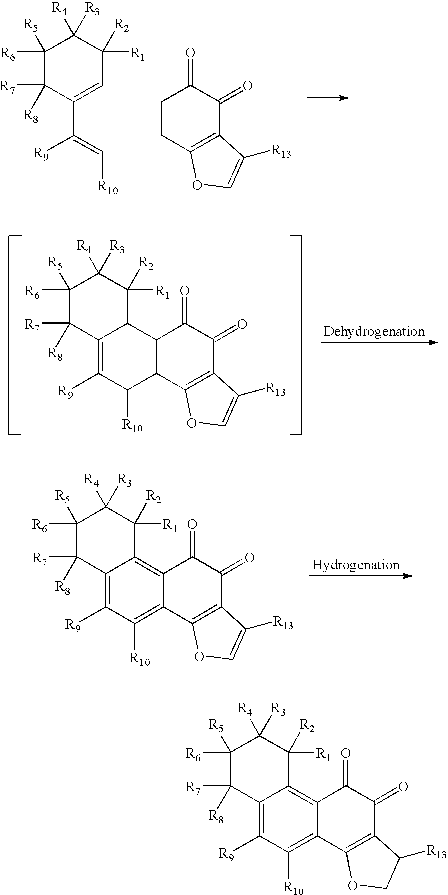

Besides the above synthetic method, compounds according to present invention in which substituents R₁₁ and R₁₂ are simultaneously hydrogen can be synthesized by the following method.

Preparation method 1 is a synthesis of active ingredient by acid-catalyzed cyclization which may be summarized in the general chemical reaction scheme as follows.

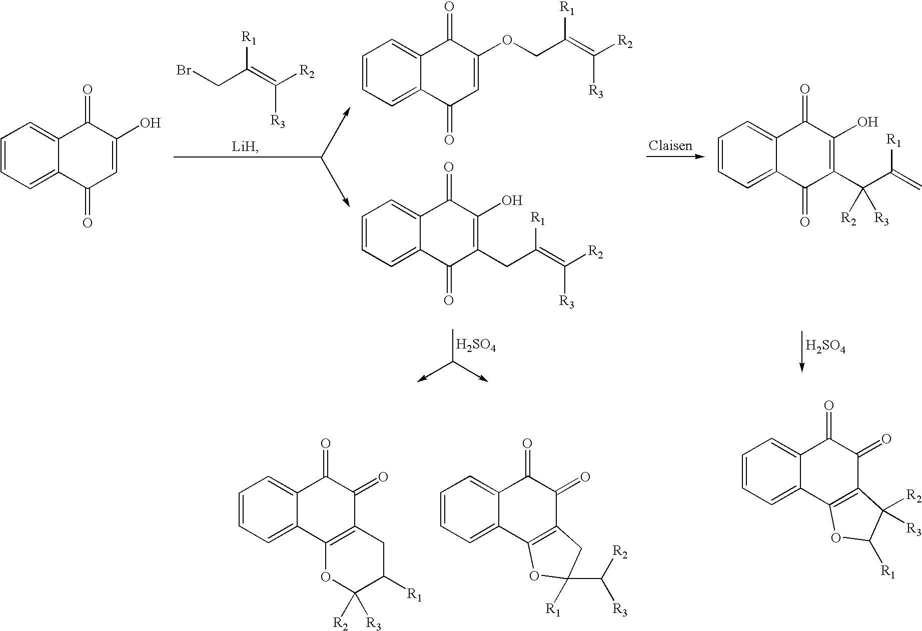

That is, when 2-hydroxy-1,4-naphthoquinone is reacted with various allylic bromides or equivalents thereof in the presence of a base, a C-alkylation product and an O-alkylation product are concurrently obtained. It is also possible to synthesize only either of two derivatives depending upon reaction conditions. Since O-alkylated derivative is converted into another type of C-alkylated derivative through Claisen Rearrangement by refluxing the O-alkylated derivative using a solvent such as toluene or xylene, it is possible to obtain various types of 3-substituted-2-hydroxy-1,4-naphthoquinone derivatives. The various types of C-alkylated derivatives thus obtained may be subjected to cyclization using sulfuric acid as a catalyst, thereby being capable of synthesizing pyrano-o-naphthoquinone or furano-o-naphthoquinone derivatives among the compounds.

Preparation method 2 is Diels-Alder reaction using 3-methylene-1,2,4-[3H]naphthalenetrione. As taught by V. Nair et al, Tetrahedron Lett. 42 (2001), 4549-4551, it is reported that a variety of pyrano-o-naphthoquinone derivatives can be relatively easily synthesized by subjecting 3-methylene-1,2,4-[3H]naphthalenetrione, produced upon heating 2-hydroxy-1,4-naphthoquinone and formaldehyde together, to Diels-Alder reaction with various olefin compounds. This method is advantageous in that various forms of pyrano-o-naphthoquinone derivatives can be synthesized in a relatively simplified manner, as compared to induction of cyclization using sulfuric acid as a catalyst.

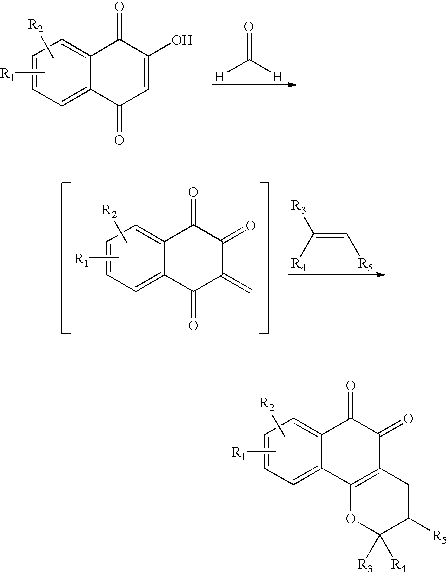

Preparation method 3 is haloalkylation and cyclization by radical reaction. The same method used in synthesis of cryptotanshinone and 15,16-dihydro-tanshinone can also be conveniently employed for synthesis of furano-o-naphthoquinone derivatives. That is, as taught by A. C. Baillie et al (J. Chem. Soc. (C) 1968, 48-52), 2-haloethyl or 3-haloethyl radical chemical species, derived from 3-halopropanoic acid or 4-halobutanoic acid derivative, can be reacted with 2-hydroxy-1,4-naphthoquinone to thereby synthesize 3-(2-haloethyl or 3-halopropyl)-2-hydroxy-1,4-naphthoquinone, which is then subjected to cyclization under suitable acidic catalyst conditions to synthesize various pyrano-o-naphthoquinone or furano-o-naphthoquinone derivatives.

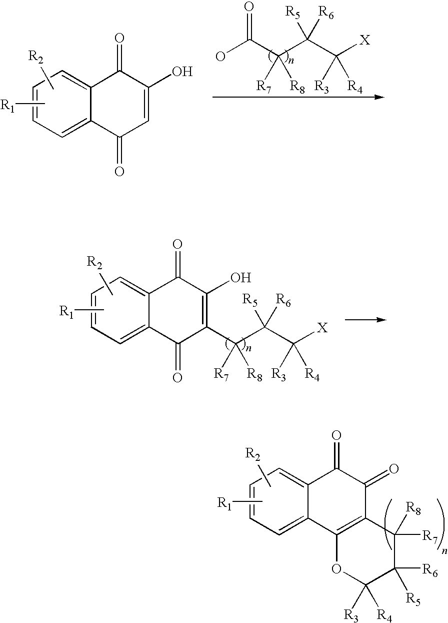

Preparation method 4 is cyclization of 4,5-benzofurandione by Diels-Alder reaction. Another method used in synthesis of cryptotanshinone and 15,16-dihydro-tanshinone may be a method taught by J. K. Snyder et al (Tetrahedron Letters 28 (1987), 3427-3430). According to this method, furano-o-naphthoquinone derivatives can be synthesized by cycloaddition via Diels-Alder reaction between 4,5-benzofurandione derivatives and various diene derivatives.

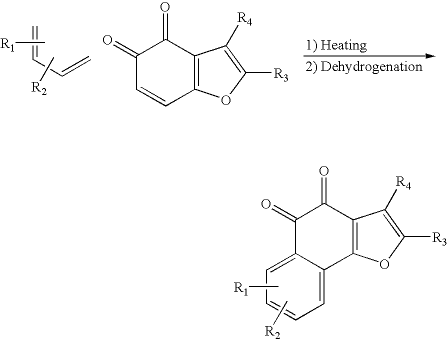

Based on the above-mentioned preparation methods, various derivatives may be synthesized using relevant synthesis methods, depending upon kinds of substituents.

Among compounds of according to the present invention, particularly preferred are in Table 1 below, but are not limited thereto.

TABLE 1

| | | | | |
|---|---|---|---|---|
| 1 | 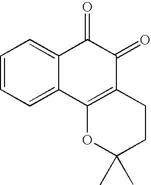 | C₁₅H₁₄O₃ | 242.27 | method 1 |
| 2 | 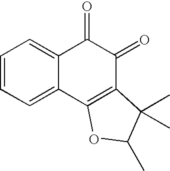 | C₁₅H₁₄O₃ | 242.27 | method 1 |
| 3 | 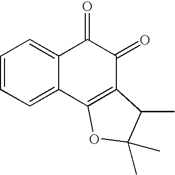 | C₁₅H₁₄O₃ | 242.27 | method 1 |
| 4 | 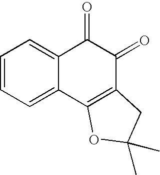 | C₁₄H₁₂O₃ | 228.24 | method 1 |
| 5 | 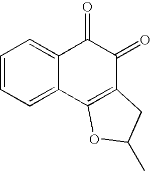 | C₁₃H₁₀O₃ | 214.22 | method 1 |
| 6 | 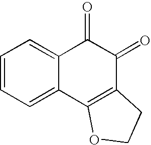 | C₁₂H₈O₃ | 200.19 | method 2 |
| 7 | 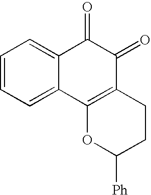 | C₁₉H₁₄O₃ | 290.31 | method 1 |
| 8 | 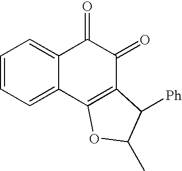 | C₁₉H₁₄O₃ | 290.31 | method 1 |
| 9 | 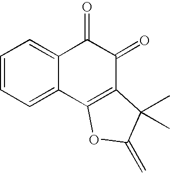 | C₁₅H₁₂O₃ | 240.25 | method 1 |
| 10 | 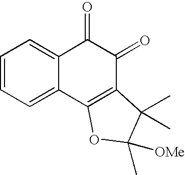 | C₁₆H₁₆O₄ | 272.30 | method 1 |
| 11 | 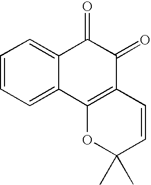 | C₁₅H₁₂O₃ | 240.25 | method 1 |
| 12 | 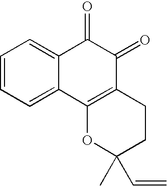 | C₁₆H₁₄O₃ | 254.28 | method 2 |
| 13 | 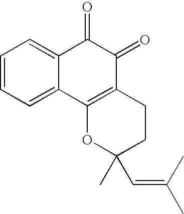 | C₁₈H₁₈O₃ | 282.33 | method 2 |
| 14 | 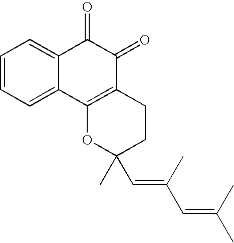 | C₂₁H₂₂O₃ | 322.40 | method 2 |
| 15 | 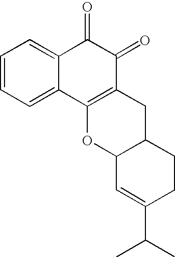 | C₂₁H₂₂O₃ | 322.40 | method 2 |
| 16 | 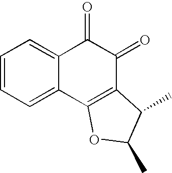 | C₁₄H₁₂O₃ | 228.24 | method 1 |
| 17 | 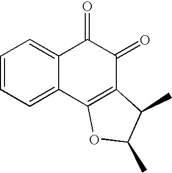 | C₁₄H₁₂O₃ | 228.24 | method 1 |
| 18 | 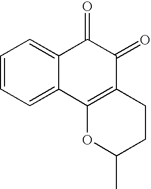 | C₁₄H₁₂O₃ | 228.24 | method 1 |
| 19 | 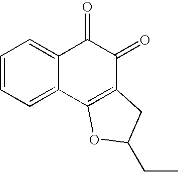 | C₁₄H₁₂O₃ | 228.24 | method 1 |
| 20 | 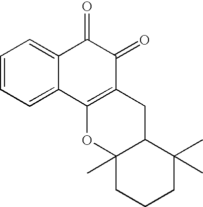 | C₂₀H₂₂O₃ | 310.39 | method 1 |
| 21 | 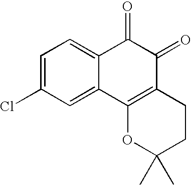 | C₁₅H₁₃ClO₃ | 276.71 | method 1 |
| 22 | 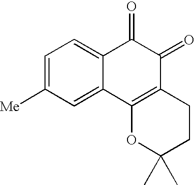 | C₁₆H₁₆O₃ | 256.30 | method 1 |
| 23 | 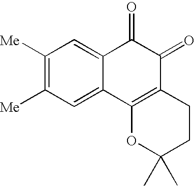 | C₁₇H₁₈O₅ | 302.32 | method 1 |
| 24 | 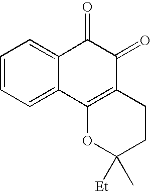 | C₁₆H₁₆O₃ | 256.30 | method 1 |
| 25 | 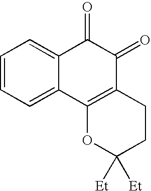 | C₁₇H₁₈O₃ | 270.32 | method 1 |
| 26 | 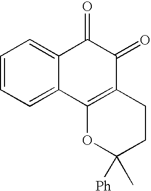 | C₂₀H₁₆O₃ | 304.34 | method 1 |
| 27 | 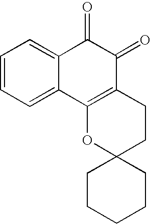 | C₁₈H₁₈O₃ | 282.33 | method 1 |
| 28 | 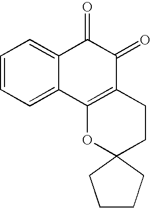 | C₁₇H₁₆O₃ | 268.31 | method 1 |
| 29 | 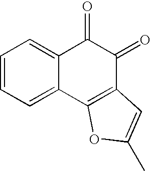 | C₁₃H₈O₃ | 212.20 | method 1 |
| 30 | 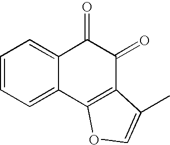 | C₁₃H₈O₃ | 212.20 | method 4 |
| 31 | 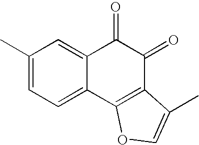 | C₁₄H₁₀O₃ | 226.23 | method 4 |
| 32 | 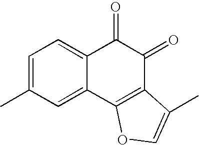 | C₁₄H₁₀O₃ | 226.23 | method 4 |
| 33 | 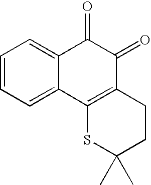 | C₁₅H₁₄O₂S | 258.34 | method 1 |
| 34 | 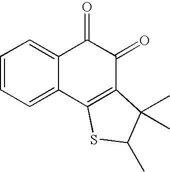 | C₁₅H₁₄O₂S | 258.34 | method 1 |
| 35 | 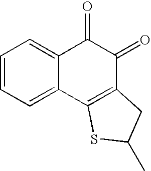 | C₁₃H₁₀O₂S | 230.28 | method 1 |
| 36 | 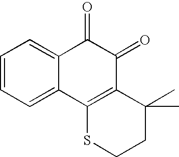 | C₁₅H₁₄O₂S | 258.34 | method 2 |
| 37 | 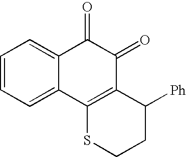 | C₁₉H₁₄O₂S | 306.38 | method 2 |
| 38 | 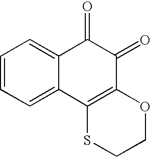 | C₁₂H₈O₃S | 232.26 | method 3 |
| 39 | 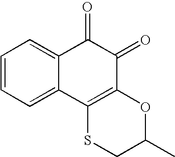 | C₁₃H₁₀O₃S | 246.28 | method 3 |
| 40 | 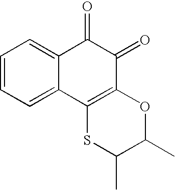 | C₁₄H₁₂O₃S | 260.31 | method 3 |
| 41 | 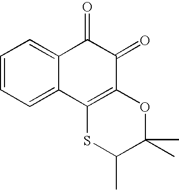 | C₁₅H₁₄O₃S | 274.34 | method 3 |
| 42 | 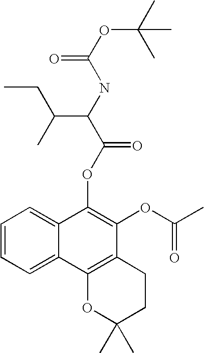 | C₂₈H₃₇O₇N | 502.22 | — |
| 43 | 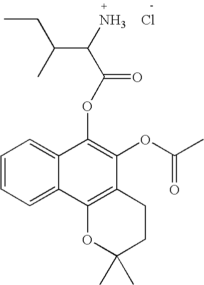 | C₂₃H₃₀O₅NCl | 940.32 | — |
| 44 | 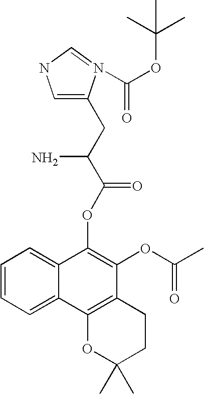 | C₂₈H₃₃O₇N₃ | 526.22 | — |
| 45 | 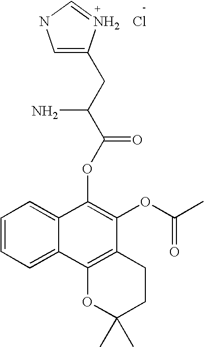 | C₂₃H₂₆O₅N₃Cl | 988.32 | — |
| 46 | 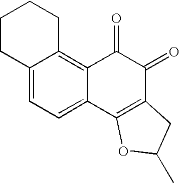 | C₁₇H₁₆O₃ | 268.31 | — |
| 47 | 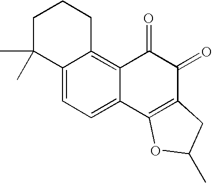 | C₁₉H₂₀O₃ | 296.36 | — |
| 48 | 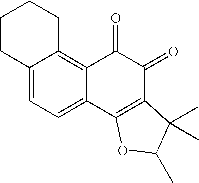 | C₁₉H₂₀O₃ | 296.36 | — |
| 49 | 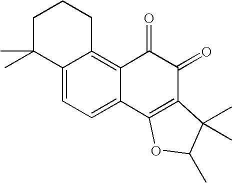 | C₂₁H₂₄O₃ | 324.41 | — |
| 50 | 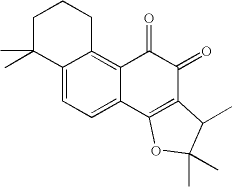 | C₂₁H₂₄O₃ | 324.41 | — |
| 51 | 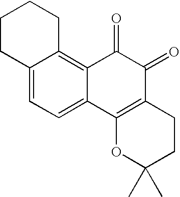 | C₁₉H₂₀O₃ | 296.36 | — |
| 52 | 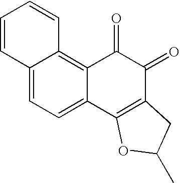 | C₁₇H₁₂O₃ | 264.28 | — |
| 53 | 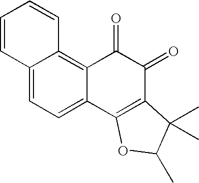 | C₁₉H₁₆O₃ | 292.33 | — |
| 54 | 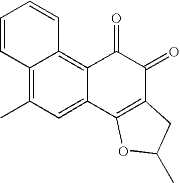 | C₁₈H₁₄O₃ | 278.30 | — |
| 55 | 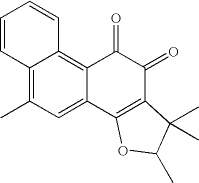 | C₂₀H₁₈O₃ | 306.36 | — |
| 56 | 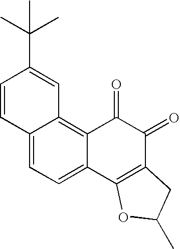 | C₂₁H₂₀O₃ | 320.38 | — |
| 57 | 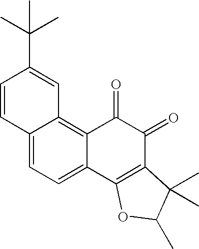 | C₂₃H₂₄O₃ | 348.43 | — |
| 58 | 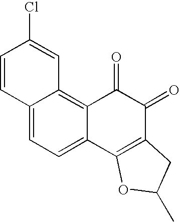 | C₁₇H₁₁ClO₃ | 298.72 | — |
| 59 | 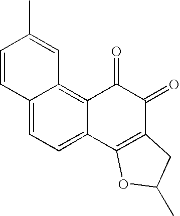 | C₁₈H₁₄O₃ | 278.30 | — |
| 60 | 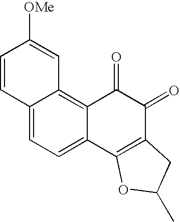 | C₁₈H₁₄O₄ | 294.30 | — |
| 61 | 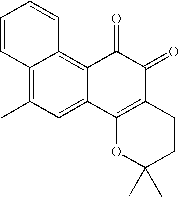 | C₂₀H₁₈O₃ | 306.36 | — |
| 62 | 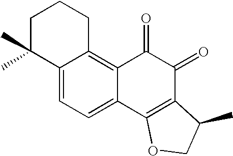 | C₁₈H₁₈O₃ | 282.33 | — |
| 63 | 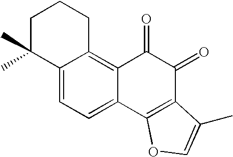 | C₁₈H₁₆O₃ | 280.33 | — |
| 64 | 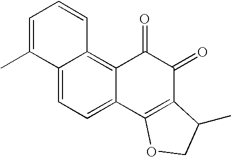 | C₁₈H₁₄O₃ | 278.33 | — |
| 65 | 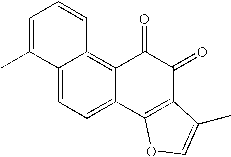 | C₁₈H₁₂O₃ | 276.33 | — |

The term “pharmaceutical composition” as used herein means a mixture of a compound of Formula 1 or Formula 2 with other chemical components, such as diluents or carriers. The pharmaceutical composition facilitates administration of the compound to an organism. Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

The term “therapeutically effective amount” means an amount of an active ingredient that is effective to relieve or reduce to some extent one or more of the symptoms of the disease in need of treatment, or to retard initiation of clinical markers or symptoms of a disease in need of prevention, when the compound is administered. Thus, a therapeutically effective amount refers to an amount of the active ingredient which exhibit effects of (i) reversing the rate of progress of a disease; (ii) inhibiting to some extent further progress of the disease; and/or, (iii) relieving to some extent (or, preferably, eliminating) one or more symptoms associated with the disease. The therapeutically effective amount may be empirically determined by experimenting with the compounds concerned in known in vivo and in vitro model systems for a disease in need of treatment.

The pharmaceutical composition of the present invention may be manufactured in a manner that is itself known, e.g., by means of conventional mixing, dissolving, granulating, dragee-making, levigating, emulsifying, encapsulating, entrapping or lyophilizing processes.

Therefore, pharmaceutical compositions for use in accordance with the present invention may be additionally comprised of a pharmaceutically acceptable carrier, a diluent or an excipient, or any combination thereof. That may be formulated in a conventional manner using one or more pharmaceutically acceptable carriers comprising excipients and auxiliaries which facilitate processing of the active compounds into preparations which can be used pharmaceutically. The pharmaceutical composition facilitates administration of the compound to an organism.

The term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffer solution is phosphate buffered saline (PBS) because it mimics the ionic strength conditions of human body fluid. Since buffer salts can control the pH of a solution at low concentrations, a buffer diluent rarely modifies the biological activity of a compound.

The compounds described herein may be administered to a human patient per se, or in the form of pharmaceutical compositions in which they are mixed with other active ingredients, as in combination therapy, or suitable carriers or excipient(s). Proper formulation is dependent upon the route of administration chosen. Techniques for formulation and administration of the compounds may be found in “Remington's Pharmaceutical Sciences,” Mack Publishing Co., Easton, Pa., 18th edition, 1990.

Various techniques of administering an active ingredient are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

Pharmaceutical composition may be formulated in a conventional manner. Preferably, the formulation may be in a form of pharmaceutically acceptable oral, parenteral, topical or membranal administration, or injection and more preferably oral administration.

The naphthoquinone-based compound is a sparingly soluble substance which dissolves a small amount (about 2 to 4%) only in solvents with excellent solubility, such as CH₂Cl₂, CHCl₃, CH₂ClCH₂Cl, CH₃CCl₃, monoglyme, or diglyme, whereas it hardly dissolves in other general polar or non-polar solvents. Therefore, despite the excellent pharmaceutical effects of the compound, there are many difficulties in formulation for the administration to a living body.

Due to the characteristics of the naphthoquinone-based compound with sparing solubility, formulation is markedly restricted. Even though the physical activities of the naphthoquinone-based compound have been revealed, the formulation form is limited to intraperitoneal or intravenous injection for administration of the compound into a body.

However, administration through injection to the patients with prostate and/or testicle (seminal glands)-related diseases, who require continuous or long term administration of the medicine, may have problems that the administration by injection accentuates pains and there may be risks of infection, pain, or hyperactivity during the administration of the medicine. Moreover, when injecting with the naphthoquinone-based compound itself or more common and simple formulation thereof, the medicine is hardly absorbed in vivo due to the characteristics of the naphthoquinone-based compound, which is a sparingly soluble medicine. That is, since the bioavailability of the compound is very low, the inherent efficacy of the medicine can not be observed.

The medicine containing the naphthoquinone-based compound as an active ingredient must be absorbed in vivo to some extent with a certain concentration or more to finally establish its pharmaceutical efficacy. However, recent researches by Jing et al. reported that the absorption rate of the quinine-based compound, i.e., cryptotanshinone, via oral administration is 2.05%, which is very low.

In this connection, it can be seen that many factors are associated with the bioavailability, that is, a level of medicine that can be used in a target tissue through absorption in vivo, after administration of the compound. The reason for this is currently known that the sparingly soluble characteristic of the medicine and first-pass problem caused by using PgP as a substrate have a great influence on the absorption (Journal of Pharmacology & Experimental Therapeutics, 23, 2006).

Therefore, in order to properly utilize inherent pharmaceutical natures of the naphthoquinone-based compounds, introducing a method capable of maximizing bioavailability is inevitable through minimizing the effects of the factors associated with bioavailability of these medicines and allowing the optimal establishment on the effects of the medicines in vivo for the treatment and prevention of prostate and/or testicle (seminal glands)-related diseases.

In this regard, the pharmaceutical composition according to the present invention, for improving sparing solubility problem and bioavailability of the naphthoquinone-based compound, may be preferably an oral pharmaceutical composition which is prepared into intestine-targeted formation.

Generally, an oral pharmaceutical composition passes through the stomach upon oral administration, is largely absorbed by the small intestine and then diffused into all the tissues of the body, thereby exerting therapeutic effects on the target tissues.

In this connection, the oral pharmaceutical composition according to the present invention enhances bioabsorption and bioavailability of a compound of Formula 1 or Formula 2 active ingredient via intestine-targeted formulation of the active ingredient. More specifically, when the active ingredient in the pharmaceutical composition according to the present invention is primarily absorbed in the stomach, and upper parts of the small intestine, the active ingredient absorbed into the body directly undergoes liver metabolism which is then accompanied by substantial degradation of the active ingredient, so it is impossible to exert a desired level of therapeutic effects. On the other hand, it is expected that when the active ingredient is largely absorbed around and downstream of the lower small intestine, the absorbed active ingredient migrates via lymph vessels to the target tissues to thereby exert high therapeutic effects.

Further, as it is constructed in such a way that the pharmaceutical composition according to the present invention targets up to the intestine which is a final destination of the digestion process, it is possible to increase the in vivo retention time of the drug and it is also possible to minimize decomposition of the drug which may take place due to the body metabolism upon administration of the drug into the body. As a result, it is possible to improve pharmacokinetic properties of the drug, to significantly lower a critical effective dose of the active ingredient necessary for the treatment of the disease, and to obtain desired therapeutic effects even with administration of a trace amount of the active ingredient. Further, in the oral pharmaceutical composition, it is also possible to minimize the absorption variation of the drug by reducing the between- and within-individual variation of the bioavailability which may result from intragastric pH changes and dietary uptake patterns.

Therefore, the intestine-targeted formulation according to the present invention is configured such that the active ingredient is largely absorbed in the small and large intestines, more preferably in the jejunum, and the ileum and intestine corresponding to the lower small intestine, particularly preferably in the ileum or colon.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or more, which is higher as compared to that of the stomach. Based on this fact, the pH-sensitive polymer may be used in order to ensure that the pharmaceutical composition reaches the lower intestinal parts without being affected by pH fluctuations of the digestive tract. Examples of the pH-sensitive polymer may include, but are not limited to, at least one selected from the group consisting of methacrylic acid-ethyl acrylate copolymer (Eudragit: Registered Trademark of Rohm Pharma GmbH), hydroxypropylmethyl cellulose phthalate (HPMCP) and a mixture thereof.

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylose, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

In the oral pharmaceutical composition according to the present invention, the active ingredient may have a crystalline structure with a high degree of crystallinity, or a crystalline structure with a low degree of crystallinity.

As used herein, the term “degree of crystallinity” is defined as the weight fraction of the crystalline portion of the total compound and may be determined by a conventional method known in the art. For example, measurement of the degree of crystallinity may be carried out by a density method or precipitation method which calculates the crystallinity degree by previous assumption of a preset value obtained by addition and/or reduction of appropriate values to/from each density of the crystalline portion and the amorphous portion, a method involving measurement of the heat of fusion, an X-ray method in which the crystallinity degree is calculated by separation of the crystalline diffraction fraction and the noncrystalline diffraction fraction from X-ray diffraction intensity distribution upon X-ray diffraction analysis, or an infrared method which calculates the crystallinity degree from a peak of the width between crystalline bands of the infrared absorption spectrum.

In the oral pharmaceutical composition according to the present invention, the crystallinity degree of the active ingredient is preferably 50% or less. More preferably, the active ingredient may have an amorphous structure from which the intrinsic crystallinity of the material was completely lost. The amorphous naphthoquinone compound exhibits a relatively high solubility, as compared to the crystalline naphthoquinone compound, and can significantly improve a dissolution rate and in vivo absorption rate of the drug.

In one preferred embodiment of the present invention, the amorphous structure may be formed during preparation of the active ingredient into microparticles or fine particles (micronization of the active ingredient). The microparticles may be prepared, for example by spray drying of active ingredients, melting methods involving formation of melts of active ingredients with polymers, co-precipitation involving formation of co-precipitates of active ingredients with polymers after dissolution of active ingredients in solvents, inclusion body formation, solvent volatilization, and the like. Preferred is spray drying. Even when the active ingredient is not of an amorphous structure, that is has a crystalline structure or semi-crystalline structure, micronization of the active ingredient into fine particles via mechanical milling contributes to improvement of solubility, due to a large specific surface area of the particles, consequently resulting in improved dissolution rate and bioabsorption rate of the active drug.

The spray drying is a method of making fine particles by dissolving the active ingredient in a certain solvent and the spray-drying the resulting solution. During the spray-drying process, a high percent of the crystallinity of the naphthoquinone compound is lost to thereby result in an amorphous state, and therefore the spray-dried product in the form of a fine powder is obtained.

The mechanical milling is a method of grinding the active ingredient into fine particles by applying strong physical force to active ingredient particles. The mechanical milling may be carried out by using a variety of milling processes such as jet milling, ball milling, vibration milling, hammer milling, and the like. Particularly preferred is jet milling which can be carried out using an air pressure, at a temperature of less than 40° C.

Meanwhile, irrespective of the crystalline structure, a decreasing particle diameter of the particulate active ingredient leads to an increasing specific surface area, thereby increasing the dissolution rate and solubility. However, an excessively small particle diameter makes it difficult to prepare fine particles having such a size and also brings about agglomeration or aggregation of particles which may result in deterioration of the solubility. Therefore, in one preferred embodiment, the particle diameter of the active ingredient may be in a range of 5 nm to 500 μm. In this range, the particle agglomeration or aggregation can be maximally inhibited, and the dissolution rate and solubility can be maximized due to a high specific surface area of the particles.

Preferably, a surfactant may be additionally added to prevent the particle agglomeration or aggregation which may occur during formation of the fine particles, and/or an antistatic agent may be additionally added to prevent the occurrence of static electricity.

If necessary, a moisture-absorbent material may be further added during the milling process. The compound of Formula 1 or Formula 2 has a tendency to be crystallized by water, so incorporation of the moisture-absorbent material inhibits recrystallization of the naphthoquinone-based compound over time and enables maintenance of increased solubility of compound particles due to micronization. Further, the moisture-absorbent material serves to suppress coagulation and aggregation of the pharmaceutical composition while not adversely affecting therapeutic effects of the active ingredient.

Examples of the surfactant may include, but are not limited to, anionic surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate. These materials may be used alone or in any combination thereof.

Examples of the moisture-absorbent material may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Some of the above-mentioned moisture absorbents may also be used as the antistatic agent.

The surfactant, antistatic agent, and moisture absorbent are added in a certain amount that is capable of achieving the above-mentioned effects, and such an amount may be appropriately adjusted depending upon micronization conditions. Preferably, the additives may be used in a range of 0.05 to 20% by weight, based on the total weight of the active ingredient.

In one preferred embodiment, during formulation of the pharmaceutical composition according to the present invention into preparations for oral administration, water-soluble polymers, solubilizers and disintegration-promoting agents may be further added. Preferably, formulation of the composition into a desired dosage form may be made by mixing the additives and the particulate active ingredient in a solvent and spray-drying the mixture.

The water-soluble polymer is of help to prevent aggregation of the particulate active ingredients, by rendering surroundings of the compound of Formula 1 or Formula 2 molecules or particles hydrophilic to consequently enhance water solubility, and preferably to maintain the amorphous state of the compound of Formula 1 or Formula 2 which is an active ingredient.

Preferably, the water-soluble polymer is a pH-independent polymer, and can bring about crystallinity loss and enhanced hydrophilicity of the active ingredient, even under the between- and within-individual variation of the gastrointestinal pH.

Preferred examples of the water-soluble polymers may include at least one selected from the group consisting of cellulose derivatives such as methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose; polyvinyl alcohols; polyvinyl acetate, polyvinyl acetate phthalate, polyvinylpyrrolidone (PVP), and polymers containing the same; polyalkene oxide or polyalkene glycol, and polymers containing the same. Preferred is hydroxypropylmethyl cellulose.

In the pharmaceutical composition of the present invention, an excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. Accordingly, the solubilizer is preferably added to maximize the solubility of the formulation by modifying physical properties of the compound of Formula 1 or Formula 2.

In this respect, the solubilizer serves to enhance solubilization and wettability of the sparingly-soluble compound of Formula 1 or Formula 2, and can significantly reduce the bioavailability variation originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug.

Preferred examples of the disintegration-promoting agent may include, but are not limited to, at least one selected from the group consisting of croscarmellose sodium, crospovidone, calcium carboxymethylcellulose, starch glycolate sodium and lower substituted hydroxypropyl cellulose. Preferred is croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

The solvent for spray drying is a material exhibiting a high solubility without modification of physical properties thereof and easy volatility during the spray drying process. Preferred examples of such a solvent may include, but are not limited to, dichloromethane, chloroform, methanol, and ethanol. These materials may be used alone or in any combination thereof. Preferably, a content of solids in the spray solution is in a range of 5 to 50% by weight, based on the total weight of the spray solution.

The above-mentioned intestine-targeted formulation process may be preferably carried out for formulation particles prepared as above.

In one preferred embodiment, the oral pharmaceutical composition according to the present invention may be formulated by a process comprising the following steps:

(a) adding the compound of Formula 1 or Formula 2 alone or in combination with a surfactant and a moisture-absorbent material, and grinding the compound of Formula 1 or 2 with a jet mill to prepare active ingredient microparticles;

(b) dissolving the active ingredient microparticles in conjunction with a water-soluble polymer, a solubilizer and a disintegration-promoting agent in a solvent and spray-drying the resulting solution to prepare formulation particles; and

(c) dissolving the formulation particles in conjunction with a pH-sensitive polymer and a plasticizer in a solvent and spray-drying the resulting solution to carry out intestine-targeted coating on the formulation particles.

The surfactant, moisture-absorbent material, water-soluble polymer, solubilizer and disintegration-promoting agent are as defined above. The plasticizer is an additive added to prevent hardening of the coating, and may include, for example polymers such as polyethylene glycol.

Alternatively, formulation of the active ingredient may be carried out by sequential or concurrent spraying of vehicles of Step (b) and intestine-targeted coating materials of Step (c) onto jet-milled active ingredient particles of Step (a) as a seed.

The oral pharmaceutical composition suitable for use in the present invention contains the active ingredient in an amount effective to achieve its intended purpose, that is therapeutic purpose. More specifically, a therapeutically effective amount refers to an amount of the compound effective to prevent, alleviate or ameliorate symptoms of disease. Determination of the therapeutically effective amount is well within the capability of those skilled in the art, especially in light of the detailed disclosure provided herein.

Further, the oral pharmaceutical composition according to the present invention is particularly useful for the treatment and/or prevention of prostate and/or testicle (seminal glands)-related diseases, as defined above. The prostate and/or testicle (seminal glands)-related diseases preferably include, but are not limited to, prostatitis or benign prostatic hyperplasia.

The term “treatment” of the disease syndromes refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing a residual amount of a naphthoquinone-based compound in the jejunum, ileum and large intestine, respectively, when single-pass intestinal perfusion was carried out according to Experimental Example 4; and

FIG. 2 is a graph showing outlet steady-state concentrations of a naphthoquinone-based compound under perfusion in Experimental Example 4; and

FIG. 3 and FIG. 4 are respectively a photograph showing prostate tissue staining of SHRs in a control group and an administration group of the compound of Example 3 in Experimental Example 7; and

FIG. 5 and FIG. 6 are respectively photographs showing prostate tissue staining of WKYs in a control group and an administration group of the compound of Example 3 in Experimental Example 7.

### DETAILED DESCRIPTION OF THE PREFERRED EMBODIMENTS

Now, the present invention will be described in more detail with reference to the following Examples. These examples are provided only for illustrating the present invention and should not be construed as limiting the scope and spirit of the present invention.

### Experimental Example 1

### Determination of Partition Coefficients

Octanol and phosphate buffer (pH 7.4) were saturated with a counter-solvent for 24 hours or more. A given amount of a naphthoquinone-based compound (compound 1 of Table 1 below) was dissolved in the thus-saturated octanol, mixed with triple-distilled water and stirred using a magnetic stirrer at 200 rpm for 13 hours or more. Samples were taken, filtered through a 0.45 μm RC Membrane filter and diluted with methanol. The diluted sample materials were analyzed by HPLC. A partition coefficient versus an amount of the compound 1 was determined. The results thus obtained are given in Table 2.

TABLE 2

| | Sample | | |
|---|---|---|---|
| | Partition Coefficient | | |
| μg/mL | 1 | 2 | 3 |
| 100 | 2.17 | 2.02 | 1.99 |
| 200 | 2.40 | 2.20 | 2.24 |
| 2000 | 2.59 | 2.62 | 2.58 |
| Excess | 2.65 | 2.05 | 2.08 |

* average partition coefficient: 2.299 (σ = 0.255)

As can be seen from Table 2, the partition coefficient was a value of 2.299, thus representing that the compound 1 is relatively fat-soluble. This result means that the compound 1 has octanol-solubility 100-fold higher than water-solubility, and sufficiently passes through a hydrophobic layer inside the cell membrane, followed by intracellular absorption.

### Example 1

### Micronization of Active Ingredient Using Jet Mill

Micronizing of an active ingredient was carried out using a Jet mill (SJ-100, Nisshin, Japan). Operation was run at a supply pressure of 0.65 Mpa, and a feed rate of 50 to 100 g/hr. 0.2 g of sodium lauryl sulfate (SLS) and 10 g of a naphthoquinone-based compound (Compound 1 of Table 1) were mixed and ground. Micronized particles were recovered and a particle size was determined by zeta potential measurement. An average particle diameter was 1500 nm.

### Example 2

### Preparation of Spray-Dried Product

The synthesized naphthoquinone-based compound (Compound 1 of Table 1) or the naphthoquinone-based compound of Example 1 (including micronized and non-micronized particles) was added to methylene chloride, and a salt such as sodium chloride, a saccharide such as white sugar or lactose, or a vehicle such as microcrystalline cellulose, monobasic calcium phosphate, starch or mannitol, a lubricant such as magnesium stearate, talc or glyceryl behenate, and a solubilizer such as Poloxamer were added to a given amount of ethanol, followed by homogeneous dispersion to prepare a spray-drying solution which will be used for subsequent spray-drying.

### Experimental Example 2

### Dissolution of Spray-Dried Formulation

To the spray-dried product of Example 2 were added approximately an equal amount of a water-soluble polymer (hydroxypropylmethyl cellulose) relative to an active ingredient, and vehicles such as Croscarmellose sodium and light anhydrous silicic acid, and the mixture was formulated without causing interference of disintegration. A drug dissolution test was carried out in a buffer (pH 6.8). All the compositions exhibited drug dissolution of 90% or higher after 6 hours.

### Experimental Example 3

### Evaluation of Relative Bioavailability of Formulations

10 male Sprague-Dawley rats were fasted, and the relative bioavailability in animals was evaluated for various formulations. Specifically, evaluation of the relative bioavailability was made for a preparation where a naphthoquinone-based compound was roughly ground and was added in conjunction with 2% by weight of sodium lauryl sulfate (SLS) to an aqueous solution (preparation prior to grinding of an active ingredient), a preparation where a naphthoquinone-based compound was ground into microparticles with a Jet mill, and was added in conjunction with 2% by weight of SLS to an aqueous solution (preparation after grinding of an active ingredient), a preparation where a formulation composed of the spray-dried product of Example 2 and the vehicle of Experimental Example 2 was added to an aqueous solution (spray-dried preparation), and a preparation where a naphthoquinone-based compound was ground into microparticles with a Jet mill, formulated using the vehicle of Experimental Example 2 and added to an aqueous solution (solid-dispersed preparation).

Randomized crossover evaluation of the bioavailability was carried out by administering 50 mg/kg of the active ingredient to each animal group. The blood concentration profiles of the active ingredient thus obtained are given in Table 3 below.

TABLE 3

| | Blood conc. (ng/mL): Fasted | | | |
|---|---|---|---|---|
| | Preparation | | | Solid- |
| Time | before | Preparation | Spray-dried | dispersed |
| (hour) | grinding | after grinding | preparation | preparation |
| 0 | 0.00 | 0.00 | 0.00 | 0.00 |
| 0.5 | 10.85 | 19.90 | 139.32 | 157.27 |
| 1 | 63.53 | 103.25 | 371.71 | 400.21 |
| 2 | 82.60 | 119.87 | 215.78 | 237.44 |
| 3 | 115.89 | 244.97 | 563.44 | 595.74 |
| 6 | 233.68 | 324.51 | 636.05 | 634.25 |
| 12 | 161.29 | 460.07 | 828.12 | 862.32 |
| 24 | 85.38 | 90.76 | 145.21 | 151.90 |
| Avg. Cmax | 233.68 | 460.07 | 828.12 | 862.32 |
| Avg. AUC | 3321.55 | 6268.01 | 11737.74 | 12151.34 |
| (last) | | | | |

As can be seen from the results of Table 3, the spray-dried formulation and the solid-dispersed formulation, which were added to an aqueous solution, exhibited an about 3-fold increase of the bioavailability in a fasted state, as compared to the comparative formulation containing the same amount of the active ingredient, particularly the formulation prior to grinding of the active ingredient.

### Experimental Example 4

### Intestinal Absorption of Compounds

In order to determine intestinal absorption (%) of a naphthoquinone-based compound, a single-pass intestinal perfusion technique was carried out in internal organs of rats, including jejunum, ileum and large intestine.

The steady-state intestinal effective permeability (P_{eff}) can be expressed according to the following equation.

P_{eff}=[−Qᵢₙ·ln (Cₒᵤₜ/Cᵢₙ)]/A

P_{eff}: Steady-state intestinal effective permeability (cm/s)

Qᵢₙ: Perfusion flow rate (0.4 mL/min)

Cᵢₙ, Cₒᵤₜ: Inlet and fluid-transport-corrected outlet solution concentrations

A: Mass transfer surface area within intestinal segment (2πrL),

r, L: Radius and length of intestinal segment

The radius (r) and length (L) of the jejunum, ileum and large intestine used in experiments are as follows: (r: jejunum, 0.21 cm; ileum, 0.22 cm; large intestine, 0.23 cm, and L: 10 cm)

The steady-state was confirmed by the ratio of the outlet to inlet concentrations (Cₒᵤₜ/Cᵢₙ) versus time. The steady-state is established when the Cₒᵤₜ/Cᵢₙ ratio of the naphthoquinone-based compound is maintained at a constant value (n=3, error bars with respect to S.D.).

Residual amounts of the naphthoquinone-based compound in the above three intestinal organs were measured at different time points. The results thus obtained are shown in FIG. 1.

As shown in FIG. 1, a relatively large amount of the naphthoquinone-based compound permeated through the intestinal tissues for the first 20 min and thereafter remained with substantially no permeation. Further, the intestinal permeability was high in the order of the large intestine, ileum and jejunum.

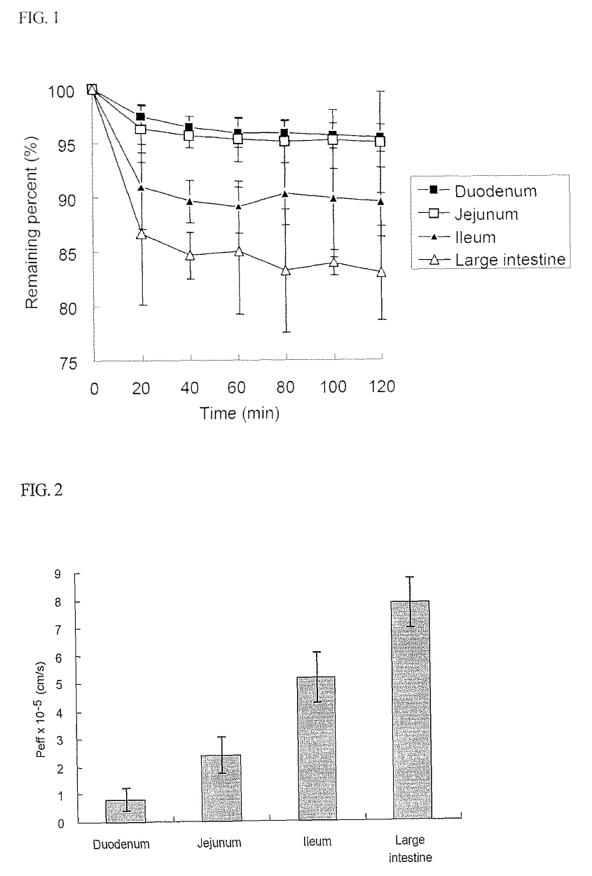

The outlet steady-state concentration of the compound under perfusion was calculated. The results thus obtained are given in Table 4 and FIG. 2, respectively. The effective permeability was measured at 4 points of each intestinal tissue. As shown in Table 4 and FIG. 2, it can be seen that the highest permeability was observed in the large intestine.

TABLE 4

| Intestinal tissues | P_{eff} × 10⁻⁵ (cm/s) |
|---|---|
| Duodenum | 0.79 ± 0.33 |
| Jejunum | 2.37 ± 1.17 |
| Ileum | 5.15 ± 1.49 |
| Large intestine | 7.82 ± 0.93 |

### Example 3

### Preparation of Intestine-Targeted Formulation

The spray-dried formulation prepared in Experimental Example 2 was added to an ethanol solution containing about 20% by weight of Eudragit S-100 as a pH-sensitive polymer and about 2% by weight of PEG #6,000 as a plasticizer, and the mixture was then spray-dried to prepare an intestine-targeted formulation.

### Experimental Example 5

### Acid Resistance of Intestine-Targeted Formulation

The intestine-targeted formulation prepared in Example 3 was exposed to pH 1.2 and pH 6.8, respectively. After 6 hours, the intestine-targeted formulation was removed and washed, and a content of an active ingredient was analyzed by HPLC. An effective amount of the active ingredient was assessed as a measure of the acid resistance. The acid resistance exhibited a very excellent result of 90 to 100%, thus suggesting that the intestine-targeted formulation is chemically stable in the stomach or small intestine.

### Experimental Example 6

### Measurement of Drug-Dissolution Profiles

After the intestine-targeted formulation was exposed to acidic environment of pH 1.2, as in Experimental Example 5, the acidity was changed to a value of pH 6.8 under artificial environment. A residual amount of the dissolved active ingredient was measured by HPLC. The results thus obtained are given in Table 5 below.

TABLE 5

| Time (min.) | Dissolution (%) at pH 6.8 |
|---|---|
| 0 | 0.00 |
| 10 | 78.05 |
| 30 | 87.57 |
| 45 | 92.13 |
| 60 | 92.27 |
| 120 | 92.66 |
| 180 | 95.61 |
| 240 | 96.29 |

### Experimental Example 7

### Therapeutic Effect of Naphthoquinone-Based Compound on Benign Prostatic Hyperplasia

8-week-old spontaneous hypertensive rats (SHRs) (essential hypertension inducing rats; Japan SLC Inc.) and Wistar-Kyoto rats (WKYs) (normal blood pressure rats; Kurea, Osaka, Japan) were used. Animals were raised in a breeding room maintained at a temperature of 23° C., 55% humidity, illumination of 300 to 500 lux, a 12-h light/dark (L/D) cycle, and ventilation of 10 to 18 times/hr. Animals were fed ad libitum pellets of Purina Rodent Laboratory Chow 5001 (purchased from Purina Mills Inc., St. Louis, Mo., USA) as a solid feed for experimental animals and tap water as drinking water. Mice were allowed to acclimate to new environment of the breeding room for two weeks and undergo preprocess for measuring blood pressure for two weeks. Then, 13-week-old SHRs and WKYs were divided into two groups, respectively, in which a first group was administered with a formulation for intestinal delivery system prepared in Example 3 according to the present invention for 10 weeks through oral route. A second group was a control group administered with saline excluding all other medicines.

10 weeks after the administration, animals of each group were anatomized and the prostates were extracted therefrom to isolate anterior lobe and posterior side lobe. The prostate membrane was completely removed, to thereby obtaining only the prostates. Samples of the anterior lobe and posterior side lobe in each group were fixed in 10% neutral formalin followed by immersing in paraffin, and hematoxylin-eosin (H&E) staining and EM examination were performed.

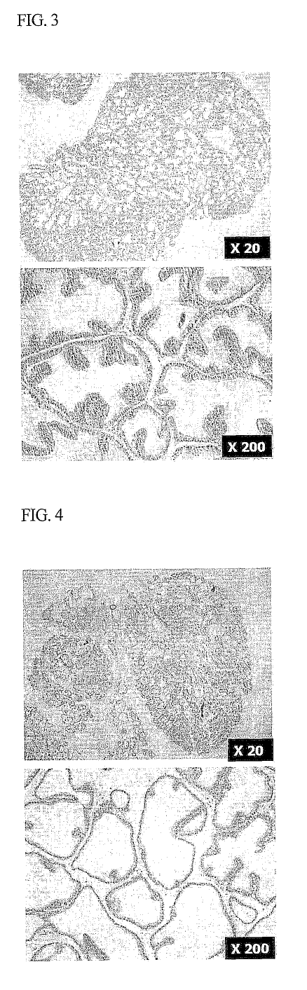

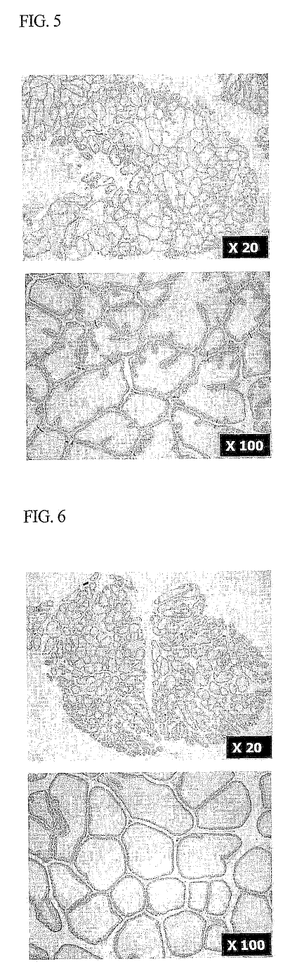

The results are shown in FIGS. 3 through 6 (FIG. 3: SHR in a control group; FIG. 4: SHR in an administration group of the compound for intestinal delivery system; FIG. 5: WKY in a control group; and FIG. 6: WKY in an administration group of the compound of Example 3).

Referring to these FIGS. 3 through 6, it was confirmed that the size of the prostate fibroblastic tissue of both SHRs and WKYs significantly reduced in the administration group of the compound for intestinal delivery system compared with the control group. Especially, it can be seen that the compound had greater effect on the treatment of benign prostatic hyperplasia of SHRs. Therefore, the compound of the present invention is believed to be useful for substantial treatment of prostate and/or testicle (seminal glands)-related diseases, such as benign prostatic hyperplasia.

### INDUSTRIAL APPLICABILITY

As apparent from the above description, a compound represented by Formula 1 or 2 according to the present invention is pharmaceutically effective for the treatment of prostate and/or testicle (seminal glands)-related diseases, especially benign prostatic hyperplasia and prostatitis. Particularly, the pharmaceutical composition for oral administration of intestinal delivery system increases absorption amount of the active ingredient and extends the duration time of the its efficacy in vivo, thereby having effects of improving pharmacokinetic properties of the drug. Consequently, by increasing availability of the naphthoquinone-compound in vivo, an excellent effect on the treatment of prostate and/or testicle (seminal glands)-related diseases, especially benign prostatic hyperplasia and prostatitis can be established.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for the treatment and/or prevention of prostate and/or testicle (seminal glands)-related diseases, comprising:
(a) a therapeutically effective amount of one or more selected from the compounds represented by Formula 1 and Formula 2 below: 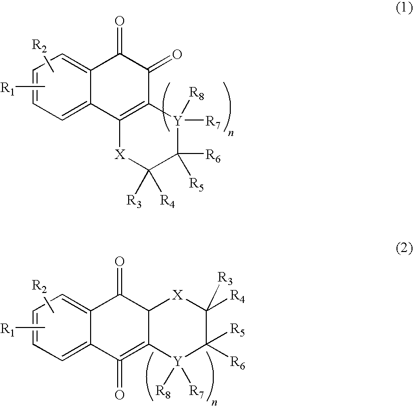
wherein
R₁ and R₂ are each independently hydrogen, halogen, hydroxyl, or C₁-C₆ lower alkyl or alkoxy, or R₁ and R₂ may be taken together to form a substituted or unsubstituted cyclic structure which may be saturated or partially or completely unsaturated;
R₃, R₄, R₅, R₆, R₇ and R₈ are each independently hydrogen, hydroxyl, amino, C₁-C₂₀ alkyl, alkene or alkoxy, C₄-C₂₀ cycloalkyl, heterocycloalkyl, aryl or heteroaryl, or two substituents of R₃ to R₈ may be taken together to form a cyclic structure which may be saturated or partially or completely unsaturated;
X is selected from a group consisting of C(R)(R′), N(R″), O and S, preferably O or S, and more preferably O, wherein R, R′ and R″ are each independently hydrogen or C₁-C₆ lower alkyl;
Y is C, S or N, with proviso that when Y is S, R₇ and R₈ are not any substituent, and when Y is N, R₇ is hydrogen or C₁-C₆ lower alkyl and R₈ are not any substituent; and
n is 0 or 1, with proviso that when n is 0, carbon atoms adjacent to n form a cyclic structure via a direct bond; or a pharmaceutically acceptable salt, prodrug, solvate or isomer thereof, and
(b) a pharmaceutically acceptable carrier, a diluent or an excipient, or any combination thereof.

2. The composition according to claim 1, wherein X is O.

3. The composition according to claim 1, wherein the prodrug is the compound represented by Formula 1a below: 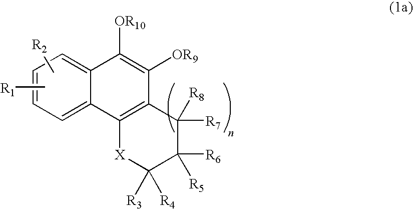
wherein,
R₁, R₂, R₃, R₄, R₅, R₆, R₇, R₈, X and n are as defined in Formula 1;
R₉ and R₁₀ are each independently —SO₃⁻Na⁺ or substituent represented by Formula A below or a salt thereof, 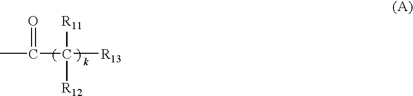
wherein,
R₁₁ and R₁₂ are each independently hydrogen or substituted or unsubstituted C₁-C₂₀ linear alkyl or C₁-C₂₀ branched alkyl
R₁₃ is selected from the group consisting of substituents i) to viii) below:
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃⁻Na⁺;
k is selected from the 0˜20, with proviso that when k is 0, R₁₁ and R₁₂ are not anything, and R₁₃ is directly bond to a carbonyl group.

4. The composition according to claim 1, wherein the compound of Formula 1 is selected from compounds of Formulas 3 and 4 below: 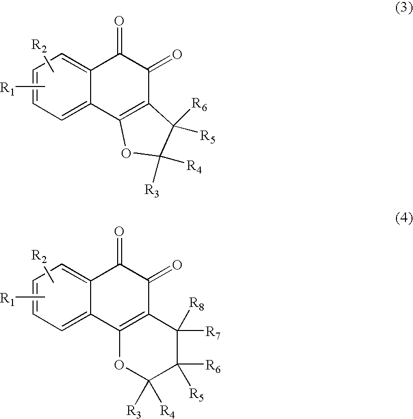
wherein, R₁, R₂, R₃, R₄, R₅, R₆, R₇ and R₈ are as defined in Formula 1.

5. The composition according to claim 4, wherein the compound of Formula 3 is selected from compounds below:

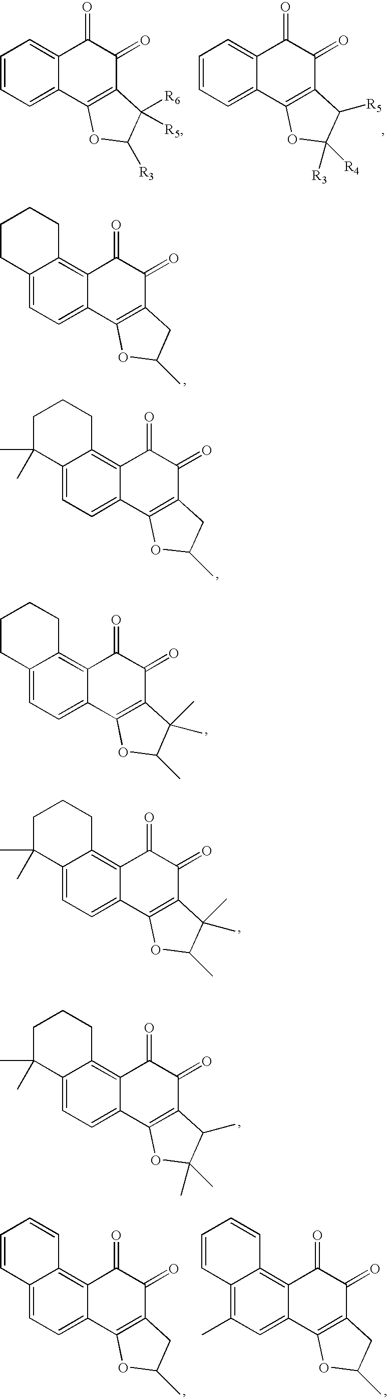

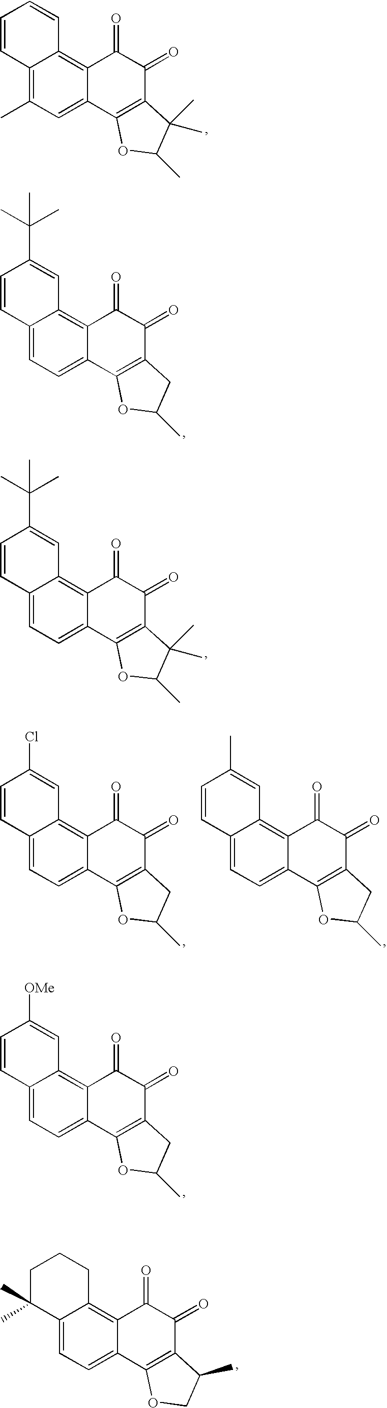

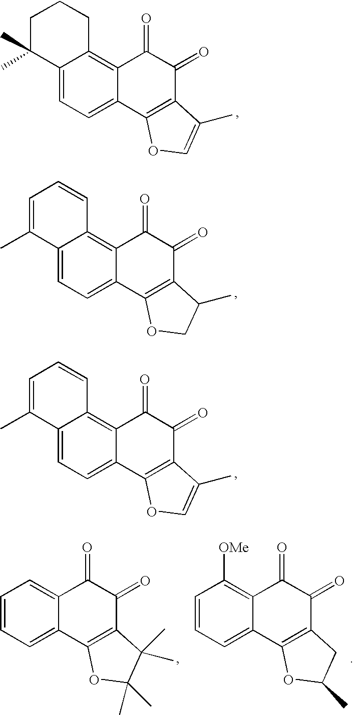

6. The composition according to claim 4, wherein the compound of Formula 4 is selected from compounds of Formulas 4a to 4c below.

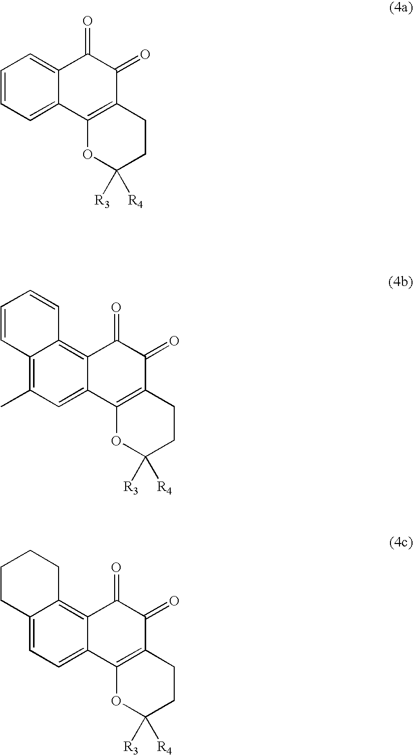

7. The composition according to claim 1, wherein the compound of Formula 2 is a compound of Formula 2a in which n is 0 and adjacent carbon atoms form a cyclic structure via a direct bond therebetween and Y is C, or a compound of Formula 2b in which n is 1 and Y is C. 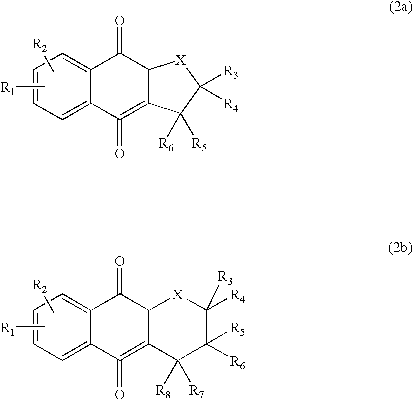
wherein, R₁, R₂, R₃, R₄, R₅, R₆, R₇, R₈ and X are as defined in Formula 2.

8. The composition according to claim 1, wherein the pharmaceutical composition is prepared into an intestine-targeted formulation.

9. The composition according to claim 8, wherein the intestine-targeted formulation is carried out by addition of a pH sensitive polymer.

10. The composition according to claim 9, wherein the pH sensitive polymer is one or more selected from the group consisting of methacrylic acid-ethyl acrylate copolymer (Eudragit: Registered Trademark of Rohm Pharma GmbH), hydroxypropylmethyl cellulose phthalate (HPMCP), and a mixture thereof.

11. The composition according to claim 9, wherein the pH sensitive polymer is added by a coating process.

12. The composition according to claim 8, wherein the intestine-targeted formulation is carried out by addition of a biodegradable polymer which is decomposable by an intestine-specific bacterial enzyme.

13. The composition according to claim 12, wherein the polymer contains an azoaromatic linkage.

14. The composition according to claim 13, wherein the polymer containing the azoaromatic linkage is a copolymer of styrene and hydroxyethylmethacrylate (HEMA).

15. The composition according to claim 12, wherein the polymer is a naturally-occurring polysaccharide or a substituted derivative thereof.

16. The composition according to claim 15, wherein the polysaccharide or substituted derivative thereof is one or more selected from the group consisting of dextran ester, pectin, amylase and ethylcellulose or pharmaceutically acceptable salt thereof.

17. The composition according to claim 8, wherein the intestine-targeted formulation is carried out by addition of a biodegradable matrix which is decomposable by an intestine-specific bacterial enzyme.

18. The composition according to claim 17, wherein the matrix is a synthetic hydrogel based on N-substituted acrylamide.

19. The composition according to claim 8, wherein the intestine-targeted formulation is carried out by a configuration with time-course release of the drug after a lag time (‘time-specific delayed-release formulation’).

20. The composition according to claim 19, wherein the time-specific delayed-release formulation is carried out by addition of a hydrogel.

21. The composition according to claim 1, wherein the compound of Formula 1 or Formula 2 is contained in a crystalline structure.

22. The composition according to claim 1, wherein the compound of Formula 1 or Formula 2 is contained in an amorphous structure.

23. The composition according to claim 1, wherein the compound of Formula 1 or Formula 2 is formulated into the form of a fine particle.

24. The composition according to claim 23, wherein the formulation for form of a fine particle is carried out by the particle micronization method selected from the group consisting of mechanical milling, spray drying, precipitation method, homogenization, and supercritical micronization.

25. The composition according to claim 24, wherein the formulation is carried out by jet milling as a mechanical milling and/or spray drying.

26. The composition according to claim 23, wherein the particle size of fine particles is within a range of 5 nm to 500 μm.

27. The composition according to claim 23, wherein one or more selected from the group consisting of surfactant, antistatic agent and moisture-absorbent is added during formation of the fine particles.

28. The composition according to claim 27, wherein the surfactant is one or more selected from the group consisting of anionic surfactants of docusate sodium and sodium lauryl sulfate; cationic surfactants of benzalkonium chloride, benzethonium chloride and cetrimide; nonionic surfactants of glyceryl monooleate, polyoxyethylene sorbitan fatty acid ester and sorbitan ester; amphiphilic polymers of polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate.

29. The composition according to claim 27, wherein the moisture-absorbent is one or more selected from the group consisting of colloidal silica, light anhydrous silicic acid, heavy anhydrous silicic acid, sodium chloride, calcium silicate, potassium aluminosilicate, and calcium aluminosilicate.

30. The composition according to claim 8, wherein during preparation of the formulation for oral administration, a water-soluble polymer, solubilizer and disintegration-promoting agent are added.

31. The composition according to claim 30, wherein the formulation is made by mixing additives and the active ingredient in the form of a fine particle in a solvent and then spray-drying the resulting mixture.

32. The composition according to claim 30, wherein the water-soluble polymer is one or more selected from the group consisting of methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose.

33. The composition according to claim 32, wherein the water-soluble polymer is hydroxypropylmethyl cellulose.

34. The composition according to claim 30, wherein the disintegration-promoting agent is one or more selected from the group consisting of Croscarmellose sodium, Crospovidone, calcium carboxymethylcellulose, starch glycolate sodium and lower substituted hydroxypropyl cellulose.

35. The composition according to claim 34, wherein the disintegration-promoting agent is Croscarmellose sodium.

36. The composition according to claim 30, wherein the solubilizer is a surfactant or amphiphile.

37. The composition according to claim 30, wherein 10 to 1000 parts by weight of the water-soluble polymer, 1 to 30 parts by weight of the disintegration-promoting agent and 0.1 to 20 parts by weight of the solubilizer are added based on 100 parts by weight of the active ingredient.

38. The composition according to claim 8, wherein the intestine-targeted formulation is prepared by a process comprising the following steps:
(a) adding the compound of Formula 1 or Formula 2 alone or in combination with a surfactant and a moisture-absorbent material, and grinding the naphthoquinone-based compound of Formula 1 or Formula 2 with a jet mill to prepare active ingredient microparticles;
(b) dissolving the active ingredient microparticles in conjunction with a water-soluble polymer, a solubilizer and a disintegration-promoting agent in a solvent and spray-drying the resulting solution to prepare formulation particles; and
(c) dissolving the formulation particles in conjunction with a pH-sensitive polymer and a plasticizer in a solvent and spray-drying the resulting solution to carry out intestine-targeted coating on the formulation particles.

39. The composition according to claim 1, wherein the Prostate-related diseases is prostatitis or benign prostatic hyperplasia (BPH).
